(19) United States
(12) Patent Application Publication
(10) Pub. No.: US 2007/0161894 A1
(43) Pub. Date: Jul. 12, 2007

(54) ULTRASOUND SYNCHRONY MEASUREMENT

(76) Inventors: Mark Zdeblick, Portola Valley, CA (US); Robert White, Stanford, CA (US)

Correspondence Address: BOZICEVIC, FIELD & FRANCIS LLP (PRTS);(PROTEUS BIOMEDICAL,INC), 1900 UNIVERSITY AVENUE, SUITE 200, EAST PALO ALTO, CA, 94303, US

(21) Appl. No.: 11/615,815

(22) Filed: Dec. 22, 2006

Related U.S. Application Data
(60) Provisional application No. 60/753,748, filed on Dec. 23, 2005.

(51) Int. Cl. A61B 8/00

(52) U.S. Cl. 600/437

(57) ABSTRACT

Methods for evaluating tissue motion of a tissue location, e.g., a cardiac location, are provided. Aspects of the methods include applying a pressure wave, e.g., ultrasound, to a subject and detecting a change in impedance between two electrodes stably associated with a target site to detect arrival of the pressure wave at said target site and thereby evaluate movement of the target site. Also provided are systems, devices and related compositions for practicing the subject methods. The subject methods and devices find use in a variety of different applications, including cardiac resynchronization therapy.

### CROSS-REFERENCE TO RELATED APPLICATIONS

Pursuant to 35 U.S.C. § 119 (e),this application claims priority to United States Provisional Application Ser. No. 60/753,748 filed on Dec. 23, 2005; the disclosure of which priority application is herein incorporated by reference.

### INTRODUCTION

### Background

In a diverse array of applications, the evaluation of tissue motion is desirable, e.g., for diagnostic or therapeutic purposes. An example of where evaluation of tissue motion is desirable is cardiac resynchronization therapy (CRT), where evaluation of cardiac tissue motion is employed for diagnostic and therapeutic purposes.

CRT is an important new medical intervention for patients suffering from heart failure, e.g., congestive heart failure (CHF). When congestive heart failure occurs, symptoms develop due to the heart's inability to function sufficiently. Congestive heart failure is characterized by gradual decline in cardiac function punctuated by severe exacerbations leading eventually to death. It is estimated that over five million patients in the United States suffer from this malady.

The aim of resynchronization pacing is to induce the interventricular septum and the left ventricular free wall to contract at approximately the same time.

Resynchronization therapy seeks to provide a contraction time sequence that will most effectively produce maximal cardiac output with minimal total energy expenditure by the heart. The optimal timing is calculated by reference to hemodynamic parameters such as dP/dt, the first time-derivative of the pressure waveform in the left ventricle. The dP/dt parameter is a well-documented proxy for left ventricular contractility.

In current practice, external ultrasound measurements are used to calculate dP/dt. Such external ultrasound is used to observe wall motion directly. Most commonly, the ultrasound operator uses the ultrasound system in a tissue Doppler mode, a feature known as tissue Doppler imaging (TDI), to evaluate the time course of displacement of the septum relative to the left ventricle free wall. The current view of clinicians is that ultrasonographic evaluation using TDI or a similar approach may become an important part of qualifying patients for CRT therapy.

A useful diagnostic imaging approach in current practice is to provide planar section views of the organ of interest, such as the heart. These views are very familiar to clinicians, and provide excellent therapeutically relevant medical information.

As currently delivered, CRT therapy is effective in about half to two-thirds of patients implanted with a resynchronization device. In approximately one-third of these patients, this therapy provides a two-class improvement in patient symptoms as measured by the New York Heart Association scale. In about one-third of these patients, a one-class improvement in cardiovascular symptoms is accomplished. In the remaining third of patients, there is no improvement or, in a small minority, a deterioration in cardiac performance. This group of patients is referred to as non-responders. It is possible that the one-class New York Heart Association responders are actually marginal or partial responders to the therapy, given the dramatic results seen in a minority.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra-cardiac stimulation, such as diaphragmatic pacing, is confirmed. With the currently available techniques, rarely is there time or means for optimizing cardiac performance.

When attempted today, CRT optimization must be preformed by laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, which is the delay between stimulation of the atria and the ventricle or ventricles themselves. These settings can be important in addition to the location of the left ventricular stimulating electrode itself in resynchronizing the patient.

More generally, CHF patients today are primarily managed on the basis of self-reported symptoms. In many cases, a patient's cardiovascular performance gradually deteriorates, with only mild subjective symptoms, until emergency admission to the hospital is required. The physician's ability to intervene early in the decompensation process—when cardiac performance is objectively declining but symptoms are not yet severe—is hampered by the lack of objective cardiac performance data characterizing the patient's condition.

A related issue is the primarily symptomatic management of patients with or without heart failure in the setting of progressive ischemic heart disease. Interventional cardiologists today have no reliable way of detecting an acute onset or worsening of cardiac ischemia when it is at an early, asymptomatic stage. If detected at this early stage, the ischemia is potentially reversible via a timely intervention. However, progressive akinesis, caused by stiffening of the cardiac muscle, is a hallmark of ischemia and is observable well before changes in the electrocardiogram (ECG) or in circulating cardiac enzymes.

Another issue is that cardiac rhythm management (CRM) systems rely upon computerized analyses of intracardiac electrograms to determine whether a pathologic arrhythmia exists and, following therapy, to characterize patients' response. Electrophysiologic-only arrhythmia detection algorithms can sometimes be confused by electrical noise and other non-cardiac interference.

It would be desirable to include objective data describing the motion of the heart to improve the reliability of such algorithms. It would be particularly useful if the data could be provided non-invasively in a doctor's office with external sensors which produce information similar to that available in a cross-sectional view, but which avoid the high radiation levels required with fluoroscopy imaging.

Relevant Literature

Publications of interest include: United States Published Patent Application Nos. 2005/0038481; 2005/0043895 and 2006/0235480; International application publication numbers W006105394; W006104869; W006113659: W003097160 and EP1503823

### SUMMARY

Methods for evaluating tissue motion of a tissue location, e.g., a cardiac location, are provided. Aspects of the methods include applying a pressure wave, e.g., ultrasound, to a subject and detecting a change in impedance between two electrodes stably associated with a target site to detect arrival of the pressure wave at said target site and thereby evaluate movement of the target site. Also provided are systems, devices and related compositions for practicing the subject methods. The subject methods and devices find use in a variety of different applications, including cardiac resynchronization therapy.

### BRIEF DESCRIPTION OF THE FIGURES

FIG. 1 provides a diagrammatic view of the positioning of the ultrasound transmitters and receivers;

FIG. 2a provides a cross sectional view of a distal lead with a quadrant electrode;

FIG. 2b is a schematic representation of three resistors;

FIG. 2c is a time-chart of the readings over a cardiac cycle;

FIG. 3a provides one example of an embodiment using conductive rubber;

FIG. 3b provides a second example of an embodiment using conductive rubber;

FIG. 3c is a schematic representation of the resistance between two electrodes.

### DETAILED DESCRIPTION

The present ultrasound synchrony measurement device provides measurement of heart synchrony using minimal modification to multiple electrode lead designs taught by some of the present inventors. Ultrasound transmissions are used to determine distance between electrodes, e.g., by using time-of-flight or continuous wave methods. The object of the ultrasound synchrony measurement device is to provide a relatively simple, robust approach to measuring the location of existing electrodes placed in or near the heart.

A special advantage of the ultrasound synchrony measurement device is that the same electrodes used in the multiple electrode lead electrodes are employed. Pacing occurs through different locations. A multiplex system is used to switch from one electrode to the other during use of the ultrasound synchrony measurement device.

In further describing various aspects of the subject invention, certain embodiments of the methods are first reviewed both in general terms and in the context of embodiments of devices and systems that may be employed to practice the methods. Following this section, representative applications in which the subject invention finds use are described, as well as other aspects of the invention, such as computer related embodiments and kits.

METHODS

As summarized above, the subject invention provides ultrasound synchrony methods for evaluating movement of a tissue location of interest, e.g. a cardiac location. Distinct from other reported concepts of heart synchrony determination, ultrasound can be broadcast at the surface of the skin at multiple locations. In one embodiment of the present invention, the ultrasound is broadcast from at least three axes. Similarly, four, five, six or more different broadcast locations can be employed as best suits the intended use or particular device configuration. In another embodiment, the entire system can be implanted, and the ultrasound can be broadcast from an internal location, such as from the “bus”, or from the pacemaker can.

In one embodiment, multiple broadcasting locations can be employed simultaneously using different frequencies, with electronic filters to separate the signals. In another embodiment, multiple broadcasting locations can be operated sequentially, in which case the same frequency can be used. For example, three broadcasting locations can be employed for determining location in three dimensional space; and more than three locations can be used to increase the resolution in three dimensional space.

The subject invention provides methods of evaluating movement of a tissue location. “Evaluating” is used herein to refer to any type of detecting, assessing or analyzing, and may be qualitative or quantitative. In certain embodiments, movement can be determined relative to another tissue location, such that the methods are employed to determine movement of two or more tissue locations relative to each other.

The tissue location(s) may be a defined location or portion of a body, i.e., subject, where in certain embodiments it is a defined location or portion (i.e., domain or region) of a body structure, such as an organ, where in certain embodiments the body structure is an internal body structure (i.e., an internal tissue location), such as an internal organ, e.g., heart, kidney, stomach, lung, etc. In certain embodiments, the tissue location is a cardiac location. As such and for ease of further description, the various aspects of the invention are now reviewed in terms of evaluating motion of a cardiac location. The cardiac location may be either endocardial or epicardial, as desired, and may be an atrial or ventricular location. Where the tissue location is a cardiac location, in certain embodiments, the cardiac location is a heart wall location, e.g., a chamber wall, such as a ventricular wall, a septal wall, etc. Although the invention is now further described in terms of cardiac motion evaluation embodiments, the invention is not so limited, the invention being readily adaptable to evaluation of movement of a wide variety of different tissue locations.

In the ultrasound synchrony measurement device, the ultrasound signal receiving electrodes are equipped with a simple, direct capacity to measure when the ultrasound energy signal from the transducer arrives at the internal electrodes, which are stably associated with a tissue location. This capacity can be provided by a number of different methods, including, but not limited to, time-of-flight and phase delay, as described in further detail below.

Aspects of the invention include applying a pressure wave to a subject, where the wave may be continuous or pulsed, e.g., as may be generated by one or more internal or external transmitters, e.g., ultrasound transmitters. The pressure wave(s) is applied to the host in a manner such that the target location, as well as sense electrodes stably associated therewith, is present in the applied pressure wave. As such, the applied pressure wave travels across the internal target location and sense electrodes.

In one embodiment of the present invention, multiple broadcasting locations can be employed simultaneously using different frequencies. For example, three broadcasting locations can be employed for determining location in three dimensional space. By using electronic filters, the signals received at each sense electrode at the target location after the arrival of the applied pressure wave can be separated into the component signals received from each separate broadcast location or axis, because each broadcast location uses a different frequency. By evaluating the signals received by the sense electrode using electronic filters, the location of the sense electrode in three dimensional space can be determined.

In another embodiment, multiple broadcasting locations can be operated sequentially, in which case the same frequency can be used. As in the previous example, three broadcasting locations can be employed for determining location in three dimensional space. The signals received at each sense electrode at the target location after the arrival of the applied pressure wave can be temporally separated into the component signals received from each separate broadcast location or axis, because each broadcast location sends a signal at a different time. By evaluating the signals received by the sense electrode, the location of the sense electrode in three dimensional space can be determined.

The ultrasound can be broadcast from at least three different axes, or similarly four, five, six or more different broadcast locations or axes can be employed. With each additional broadcast location, or axis, used, the location of the sense electrodes can be refined to increase the resolution of the inventive method.

The arrival of the applied pressure wave at the target location is determined by detecting a change in impedance between the sense electrodes stably associated with the target location.

By “stably associated with” is meant that the sensing element is substantially if not completely fixed relative to the tissue location of interest such that when the tissue location of interest moves, the sensing element also moves. As the sensing element is stably associated with the tissue location, its movement is at least a proxy for, and in certain embodiments is the same as, the movement of the tissue location to which it is stably associated, such that movement of the ultrasound sensing element can be used to evaluate movement of the tissue location of interest. The ultrasound sensing element may be stably associated with the tissue location using any convenient approach, such as by attaching the sensing element to the tissue location by using an attachment element, such as a hook, etc., by having the sensing element on a structure that compresses the sensing element against the tissue location such that the two are stably associated, etc. In a given embodiment, the sensing element can provide output in an interval fashion or continuous fashion for a given duration of time, as desired.

In certain embodiments, a single pair of sense electrodes (collectively referred to herein as a sense element) is employed. In such methods, evaluation may include monitoring movement of the tissue location over a given period of time. In certain embodiments, two or more distinct sensing elements are employed to evaluate movement of two or more distinct tissue locations. The number of different sensing elements that are employed in a given embodiment may vary greatly, where in certain embodiments the number employed is 2 or more, such as 3 or more, 4 or more, 5 or more, 8 or more, 10 or more, etc. In such multi-sensor embodiments, the methods may include evaluating movement of the two or more distinct locations relative to each other.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

The tissue movement evaluation data obtained using the subject methods may be employed in a variety of different applications, including but not limited to monitoring applications, treatment applications, etc. Certain applications in which the data obtained from the subject methods finds use are further reviewed in greater detail below.

The capacity to measure when the ultrasound energy signal from the transducer arrives at the internal electrodes can be provided by a number of different methods, including, but not limited to, time-of-flight and phase delay. In one embodiment of the ultrasound synchrony measurement device, the ultrasound transmission signal is provided in a pulsed form. In this case, the time-of-flight from when the pulse is emitted by the ultrasound transmitter to when it is received by the electrode on the lead is used to determine the distance between each electrode and the transmitter. The time-of-flight can be used to calculate the distance between the two electrodes. From this information, synchrony measurement is derived.

In the case of ultrasound transmissions, the speed of the transmission is the speed of sound. This is an advantage over other transmission frequencies in that the flight time is readily discerned. The velocity relative to the radial direction from the broadcaster is easily determined by measuring the time between two pulses. As relative velocity changes, the time between successive pulses from a constant source changes; because of the Doppler effect, a shorter time indicates that objects are approaching each other, while a longer time indicates that objects are moving away from each other. Approaches to assess time-of-flight are taught in a US patent provisional application by the present inventor and colleagues, 11/249,152 filed Oct. 11, 2005, incorporated in its entirety herein.

In an additional embodiment of the present invention, a continuous wave is broadcast by the ultrasound transmitters. The phase lag of when the ultrasound transmission is received by the electrode is related to the distance between the transmitter and electrode pair or satellite. From this information, synchrony measurement is derived. Continuous wave methods are described in International application No. PCT/US2005/036035 filed on Oct. 6, 2005, incorporated in its entirety herein.

A very simple modification to existing multiple electrode lead designs can be employed to enable the unique function of the present invention, as described below. Such a multiple electrode lead design is taught by the present inventor in PCT application No. 11/219,305 entitled Methods And Apparatus For Tissue Activation And Monitoring filed Sep. 01, 2005 Attorney Docket No. 021308-001340US incorporated in its entirety herein.

Ultrasound Synchrony Measurement

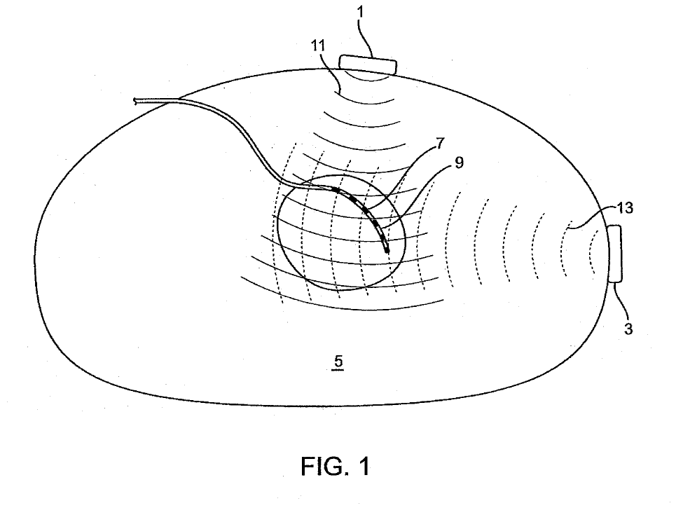

FIG. 1 describes the overall setup of the ultrasound transmitters 1 and 3 in the ultrasound synchrony measurement device. Ultrasound transmitters 1 and 3 are shown in the cross section of the patient's torso 5. An arbitrary array 7 of satellites, each comprising one or more electrodes is provided on a lead 9.

Ultrasound transmitters 1 and 3 emit ultrasound signal 11 and 13, respectively. The distance between ultrasound transmitters 1 and 3 and each satellite in the array of satellites 7 is related to the time it takes for the ultrasound signals 11 and 13 to reach each satellite in the array of satellites 7.

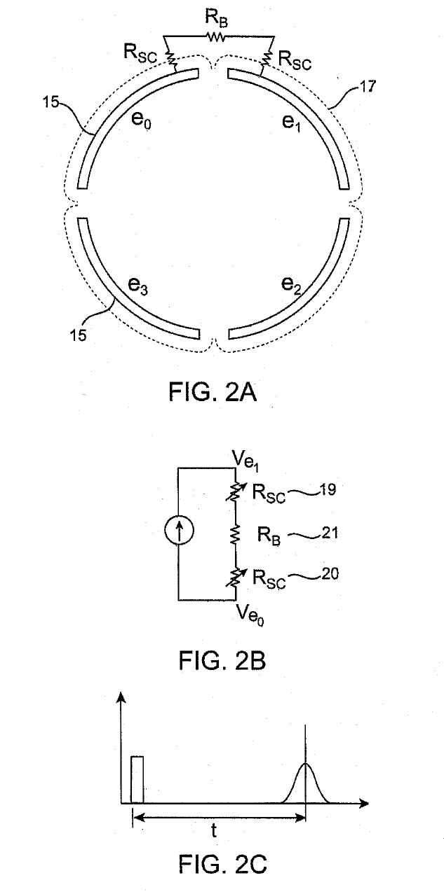

FIGS. 2a-c describes a basic, simplified implementation of how ultrasound energy arrival time is measured by the ultrasound synchrony measurement device. Shown in FIG. 2a is a cross section of distal lead developed by the inventor and colleagues, including a quadrant electrode, taught by the inventor and colleagues in US provisional patent No. 60/638692 entitled “High Fatigue Life Semiconductor Electrodes” filed Dec. 22, 2004, attorney docket number 021308-002000US incorporated by reference in its entirety herein.

The four electrodes 15 within the quadrant electrode section are spaced regularly around the circumference of the lead cross-section. The resistance between two of these adjacent electrodes is related to the bulk conductance (R_{B}). This resistance (R_{SC}). is also the electrical conductivity of the boundary layer 17, which could also be considered a space charge region.

Boundary layer 17 is a very narrow, thin region between the electrode and the bulk. The boundary layer has the largest impedance. The boundary layer impedance is typically on the order of hundreds of ohms, and often as high as a thousand ohms.

The ultrasound pressure modulates the thickness of electrical boundary layer 17. This modulation changes the impedance. Using the electrical lead described previously by the present inventor and colleagues, the arrival of an acoustic energy pulse is determined. This measurement is provided because the acoustic energy pulse modulates the impedance between two of the four electrodes 15.

FIG. 2b is a simple schematic representation of three resistors which model the impedance between two electrodes in one embodiment of the ultrasound synchrony measurement device. The three resistors include two boundary layer resistors 19 and 20. Boundary layer resistors 19 and 20 serve as space charge resistors between one of the electrodes 15 and the bulk resistor. A bulk resistor 21 connects the boundary layer resistors 19 and 20. Thus the resistance between the two electrodes is a function of the pressure.

FIG. 2c is a time-chart of the data output from the ultrasound synchrony measurement device. The vertical axis represents either pressure or voltage. The horizontal axis represents time.

In use of the ultrasound synchrony measurement device, a pressure pulse is broadcast near time zero. Subsequently, at time T, a voltage is measured.

The changing impedance is measured between electrode e0 and e1. This change in impedance corresponds to the time it took for that pulse to arrive at the electrodes.

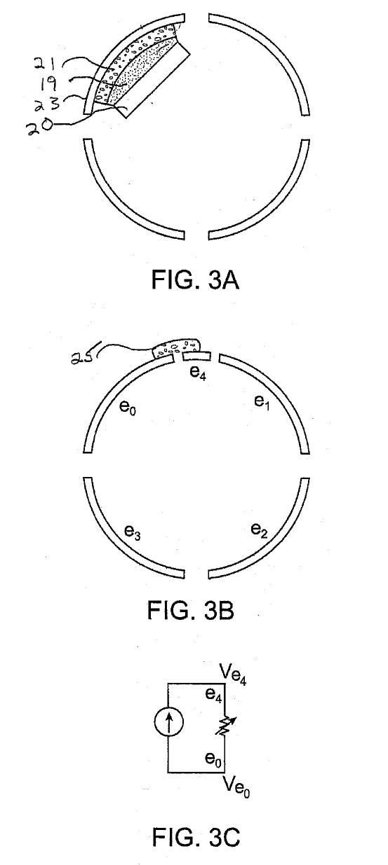

FIG. 3a-c is another embodiment of the design described above. In the prior case, the change in the impedance of the boundary layer was employed. In the current embodiment, a conductive rubber is incorporated into the design. This material can be selected from a number of different sources. By example, Zoflex and similar materials are suitable in this regard.

ZOFLEX® FL-45 is used to produce flexible conductive parts and is a conductive adhesive. The ZOFLEX® FL-45 is a two-part system which includes a liquid conductive rubber that that cures at room temperature to a Shore A hardness of 45 with very low resistance. It has a mix ratio, by weight, of 6.6A to 1B, a pot life of 15 min at 25° C. (77° F.), a viscosity (when mixed) of 25,000 cps, a specific gravity of 1.7, a volume resistivity of 0.2 ohm·cm, and a shore a hardness of 45. Other appropriate materials best suited to a specific embodiment of the ultrasound synchrony measurement device will be recognized and easily selected by the ordinary skilled artisan.

The conductivity of conductive rubber changes with pressure. As it is squeezed, conductive rubber become more conductive. As a result, its resistance will drop. Several different approaches to the use of conductive rubber in the ultrasound synchrony measurement device are useful.

FIG. 3a shows the backside of the integrated circuit 20. One platinum contact 19 is provided to the conductive rubber 21. One of the electrodes 23 becomes the other contact to the conductive rubber.

The contact moves in and out ever so slightly with the ultrasound energy. This movement causes a change in impedance between the electrode on the chip and the electrode that faces the blood. In the case where five electrodes are provided on the IC chip, the fifth electrode is allocated to provide that capacity.

FIG. 3b shows a different approach. Five electrodes are placed near the surface. A drop of conductive rubber 25 is positioned between a relatively small electrode and one of the nearby electrodes. Impedance between e4, a relatively small electrode, and e0, an adjacent electrode, is measured. Impedance is then proportional to pressure. That signal is then used to measure time-of-flight between any of the ultrasound transmitters and each of these electrodes. Triangulation provides a determination of the change in position of the electrodes. FIG. 3b is a simple, schematic representation of the resistance between two electrodes in this embodiment of the ultrasound synchrony measurement device.

Systems

Aspects of the invention include systems, including implantable medical devices and systems, which include the devices of the invention and can be employed to practice methods according to the invention, e.g., as described above. The systems may also be configured to perform a number of different functions, including but not limited to electrical stimulation applications, e.g., for medical purposes, such as pacing, CRT, etc.

The systems may have a number of different components or elements. Elements that are present in the systems may include a sensing element, such as an implantable receive electrode, stably associated with a target location, a pressure wave transmitter such as an ultrasound transmitter, and a signal processing element configured to evaluate data obtained from the sensing element to detect the arrival of a pressure wave at the target location. The system may also include a processing program for practicing the methods, where the programming may be implemented in an implanted or external processor, e.g., as described above.

In certain embodiments of the subject systems, one or more receive electrodes of the invention are electrically coupled to at least one elongated conductive member, e.g., an elongated conductive member present in a lead, such as a cardiovascular or vascular lead. In certain embodiments, the elongated conductive member is part of a multiplex lead, e.g., as described in Published PCT Application No. WO 2004/052182 and U.S. patent application Ser. No. 10/734,490, the disclosure of which is herein incorporated by reference. In some embodiments of the invention, the devices and systems may include onboard logic circuitry or a processor, e.g., present in a central control unit, such as a pacemaker can. In these embodiments, the central control unit may be electrically coupled to one or more receive electrodes via one or more conductive members.

In certain embodiments of the subject systems, one or more sets of electrodes are electrically coupled to at least one elongated conductive member, e.g., an elongated conductive member present in a lead, such as a cardiovascular or vascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or fewer wires, such as only 2 wires or only 1 wire. Multiplex lead structures of interest include those described in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; No. 60/793,295 titled “High Phrenic, Low Pacing Capture Threshold Implantable Addressable Segmented Electrodes” filed on Apr. 18, 2006 and No. 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

In certain embodiments, the receive electrodes are segmented electrode structures. By segmented electrode structure is meant an electrode structure that includes two or more, e.g., three or more, including four or more, disparate electrode elements. Embodiments of segmented electrode structures are disclosed in application Ser. Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; No. 60/793,295 titled “High Phrenic, Low Pacing Capture Threshold Implantable Addressable Segmented Electrodes” filed on Apr. 18, 2006 and No. 60/807,289 titled “High Phrenic, Low Capture Threshold Pacing Devices and Methods,” filed Jul. 13, 2006; the disclosures of the various segmented electrode structures of these applications being herein incorporated by reference.

In certain embodiments, the receive electrodes are “addressable” electrode structures. Addressable electrode structures include structures having one or more electrode elements directly coupled to control circuitry, e.g., present on an integrated circuit (IC). Addressable electrode structures include satellite structures that include one more electrode elements directly coupled to an IC and configured to be placed along a lead. Examples of addressable electrode structures that include an IC are disclosed in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; No. 60/793,295 titled “High Phrenic, Low Pacing Capture Threshold Implantable Addressable Segmented Electrodes” filed on Apr. 18, 2006 and No. 60/807,289 titled “High Phrenic, Low Capture Threshold Pacing Devices and Methods,” filed Jul. 13, 2006; the disclosures of the various addressable electrode structures of these applications being herein incorporated by reference.

Embodiments of the subjects systems may incorporate one or more effector elements. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, AC voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Use of the systems may include visualization of data obtained with the devices. Some of the present inventors have developed a variety of display and software tools to coordinate multiple sources of sensor information which will be gathered by use of the inventive systems. Examples of these can be seen in international PCT application Ser. No. PCT/US2006/012246; the disclosure of which application, as well as the priority applications thereof are incorporated in their entirety by reference herein.

Data obtained in accordance with the invention, as desired, can be recorded by an implantable computer. Such data can be periodically uploaded to computer systems and computer networks, including the Internet, for automated or manual analysis.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

Aspects of the invention include systems, including implantable medical devices and systems, which include the devices of the invention and can be employed to practice methods according to the invention, e.g., as described above. The systems may also be configured to perform a number of different functions, including but not limited to electrical stimulation applications, e.g., for medical purposes, such as pacing, CRT, etc.

Utility

The ultrasound synchrony measurement method of evaluating tissue location movement finds use in a variety of different applications. As indicated above, one application of the subject invention is for use in cardiac resynchronization therapy (CRT)(i.e., biventricular pacing). CRT remedies the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected. Using the subject ultrasound synchrony measurement approach, the electromechanical delay of the left lateral ventricle can be evaluated and the resultant data employed in CRT, e.g., using the approaches reviewed above and/or known in the art and reviewed at Col. 22, lines 5 to Col. 24, line 34 ff of U.S. Pat. No. 6,795,732, the disclosure of which is herein incorporated by reference.

In a fully implantable system the location of the pacing electrodes on multi electrode leads and pacing timing parameters are continuously optimized by the pacemaker. The subject methods and devices can be used to determine the cardiac wall motion and timing of cardiac wall motion of a first cardiac wall (e.g. the interventricular septum) relative to a second cardiac wall (e.g. the free wall of the left ventricle) to detect ventricular mechanical dyssynchrony. The pacemaker can then determine the location and parameters which minimize intraventricular dyssynchrony, interventricular dyssynchrony, or electromechanical delay of the left ventricle lateral wall in order to optimize CRT. This cardiac wall motion sensing system can also be used during the placement procedure of the cardiac leads in order to optimize CRT. An external controller could be connected to the cardiac leads and a skin patch electrode during placement of the leads. The skin patch acts as the reference electrode until the pacemaker is connected to the leads. In this scenario, for example, the optimal left ventricle cardiac vein location for CRT is determined by acutely measuring intraventricular dyssynchrony.

The subject methods and devices can be used to adjust a resynchronization pacemaker either acutely in an open loop fashion or on a nearly continuous basis in a closed loop fashion.

Other uses for this system are as an ischemia detector. It is well understood that in the event of acute ischemic events one of the first indications of such ischemia is akinesis, i.e., decreased wall motion of the ischemic tissue as the muscle becomes stiffened. A wall motion system would be a very sensitive indicator of an ischemic process, by ratio metrically comparing the local wall motion to a global parameter such as pressure; this has been previously described in another Proteus patent. One can derive important information about unmonitored wall segments and their potential ischemia. For example, if an unmonitored section became ischemic, the monitored segment would have to work harder and have relatively greater motion in order to maintain systemic pressure and therefore ratio metric analysis would reveal that fact.

Another application of such position indicators that record wall motion is as a superior arrhythmia detection circuit. Current arrhythmia detection circuits rely on electrical activity within the heart. Such algorithms are therefore susceptible to confusing electrical noise for an arrhythmia. There is also the potential for misidentifying or mischaracterizing arrhythmia based on electrical events when mechanical analysis would reveal a different underlying physiologic process. Therefore the current invention could also be adapted to develop a superior arrhythmia detection and categorization algorithm.

Additional applications in which the subject invention finds use include, but are not limited to: the detection of electromechanical dissociation during pacing or arrhythmias, differentiation of hemodynamically significant and insignificant ventricular tachycardias, monitoring of cardiac output, mechanical confirmation of capture or loss of capture for autocapture algorithms, optimization of multi-site pacing for heart failure, rate responsive pacing based on myocardial contractility, detection of syncope, detection or classification of atrial and ventricular tachyarrhythmias, automatic adjustment of sense amplifier sensitivity based on detection of mechanical events, determination of pacemaker mode switching, determining the need for fast and aggressive versus slower and less aggressive anti-tachyarrhythmia therapies, or determining the need to compensate for a weakly beating heart after therapy delivery (where these representative applications are reviewed in greater detail in U.S. Pat. No. 6,795,732, the disclosure of which is herein incorporated by reference), and the like.

In certain embodiments, the subject invention is employed to overcome barriers to advances in the pharmacologic management of CHF, which advances are slowed by the inability to physiologically stratify patients and individually evaluate response to variations in therapy. It is widely accepted that optimal medical therapy for CHF involves the simultaneous administration of several pharmacologic agents. Progress in adding new agents or adjusting the relative doses of existing agents is slowed by the need to rely solely on time-consuming and expensive long-term morbidity and mortality trials. In addition, the presumed homogeneity of clinical trial patient populations may often be erroneous since patients in similar symptomatic categories are often assumed to be physiologically similar. It is desirable to provide implantable systems designed to capture important cardiac performance and patient compliance data so that acute effects of medication regimen variation may be accurately quantified. This may lead to surrogate endpoints valuable in designing improved drug treatment regimens for eventual testing in longer-term randomized morbidity and mortality studies. In addition, quantitative hemodynamic analysis may permit better segregation of drug responders from non-responders thereby allowing therapies with promising effects to be detected, appropriately evaluated and eventually approved for marketing. The present invention allows for the above. In certain embodiments, the present invention is used in conjunction with the Pharma-informatics system, as described in PCT Application Serial No. PCT/US2006/016370 filed on Apr. 28, 2006; the disclosure of which is herein incorporated by reference.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that this has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

Computer Readable Medium

One or more aspects of the subject invention may be in the form of computer readable storage medium having a processing program stored thereon for implementing the subject methods. The computer readable medium may be, for example, in the form of a computer disk or CD, a floppy disc, a magnetic “hard card”, a server, or any other computer readable storage medium capable of containing data or the like, stored electronically, magnetically, optically or by other means. Accordingly, the stored processing program would operate a processor,embodying steps for carrying out the subject methods that may be transferred or communicated to a processor, e.g., by using a computer network, server, or other interface connection, e.g., the Internet, or other relay means.

More specifically, the computer readable storage medium may include a stored processing program embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable storage mediums such that the systems are configured to practice the subject methods.

Kits

As summarized above, also provided are kits for use in practicing the subject methods. The kits at least include a computer readable storage medium, as described above. The computer readable storage medium may be a component of other devices or systems, or components thereof, in the kit, such as a processor, an adaptor module, a pacemaker, etc. The kits and systems may also include a number of optional components that find use with the subject energy sources, including but not limited to, segmented electrode structures, implantation devices, etc. The segmented electrode structures may be present on a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1. A method for detecting the arrival of an applied pressure wave at an internal target site in a subject, said method comprising:
(a) applying a pressure wave to said subject; and
(b) detecting a change in impedance between two electrodes stably associated with said target site to detect arrival of said pressure wave at said target site.

2. The method according to claim 1, wherein said pressure wave is a sonic wave.

3. The method according to claim 2, wherein said sonic wave is applied from at least one location.

4. The method according to claim 3, wherein said sonic wave is applied from two or more locations.

5. The method according to claim 3, wherein said location is an internal location.

6. The method according to claim 3, wherein said location is an external location.

7. The method according to claim 2, wherein said sonic wave is applied from an ultrasound transmitter.

8. The method according to claim 2, wherein said sonic wave is in a continuous wave form.

9. The method according to claim 1, wherein said sonic wave is in a pulsed form.

10. The method according to claim 1, wherein said applying is from multiple locations broadcasting in a sequential manner.

11. The method according to claim 1, wherein said applying is from multiple locations broadcasting simultaneously using different frequencies.

12. The method according to claim 1, wherein said two electrodes are part of a segmented electrode structure.

13. The method according to claim 12, wherein said segment electrode structure is present on a multiplex lead.

14. The method according to claim 13, wherein said segmented electrode structure is a quadrant electrode.

15. The method according to claim 1, wherein said two electrodes are coupled to each other by a conductive material.

16. The method according to claim 15, wherein said conductive material is a conductive rubber.

17. The method according to claim 1, wherein said tissue location is a cardiac location.

18. The method according to claim 17, wherein said cardiac location is a heart wall location.

19. The method according to claim 1, wherein said method is a method of evaluating cardiac wall motion.

20-23. (canceled)

24. A system for detecting the arrival of a pressure wave at an internal target site, said system comprising:
(a) a pressure wave transmitter;
(b) a sensing elements stably associated with said target site, wherein said sensing element comprises two spaced apart electrodes; and
(c) a signal processing element configured to evaluate data obtained from said sensing element to detect the arrival of a pressure wave at said target location.

25-34. (canceled)
